(19) United States
(12) Patent Application Publication
(10) Pub. No.: US 2024/0010643 A1
(43) Pub. Date: Jan. 11, 2024

(54) PREPARATION METHOD OF SUBSTITUTED PYRIMIDINE PIPERAZINE COMPOUNDS

(71) Applicant: SUNSHINE LAKE PHARMA CO., LTD., Dongguan, Guangdong (CN)

(72) Inventors: Chuanfei JIN, Dongguan (CN); Haiping LIANG, Dongguan (CN); Tengfei XU, Dongguan (CN); Yingjun ZHANG, Dongguan (CN)

(73) Assignee: SUNSHINE LAKE PHARMA CO., LTD., Dongguan, Guangdong (CN)

(21) Appl. No.: 18/026,509

(22) PCT Filed: Sep. 16, 2021
(86) PCT No.: PCT/CN2021/118629

§ 371 Date: Mar. 15, 2023

(30) Foreign Application Priority Data
Sep. 18, 2020 (CN) 202010986938.0

(51) Int. Cl. C07D 417/14

(52) U.S. Cl. CPC C07D 417/14

(57) ABSTRACT

A preparation method of substituted pyrimidine piperazine compounds, as well as intermediate compounds used in the method. The preparation method has the advantages of cheap and easily available raw materials, mild conditions, short reaction time, safety and controllability, high total yield, which is especially suitable for industrial production.

### CROSS-REFERENCE TO RELATED APPLICATION

This application claims the priority and benefits of Chinese Patent Application No. 202010986938.0, filed with the State Intellectual Property Office of China on Sep. 18, 2020, which is incorporated herein by reference in its entirety.

### FIELD

The present invention relates to the field of medicinal chemistry, in particular to a preparation method of substituted pyrimidine piperazine compounds that can be used as reuptake inhibitors and/or 5-HT_{1A} receptor agonists.

### BACKGROUND

5-hydroxytryptamine (5-HT), a neurotransmitter that transmits signals in the brain and nervous system, and plays an important role in central nervous system (CNS) dysfunction, especially in anxiety, depression, aggression, and impulsive emotions. Antagonizing or agonizing certain types of 5-hydroxytryptamine receptors can effectively regulate central nervous system dysfunction. The 5-HT_{1A} receptor is a G-protein-coupled receptor widely distributed in regions that receive 5-hydroxytryptamine input from the raphe nuclei; activation of the presynaptic 5-HT_{1A} receptor may also indirectly reduce 5-hydroxytryptamine transmission through the inhibition of tyrosine hydroxylase synthesis, as well as the activity of glutamatergic pathway that originates in the medial prefrontal cortex and projects to the raphe nuclei (Jonathan Savitz, Irwin Lucki, Wayne C. Drevets. 5-HT_{1A} receptor function in major depressive disorder. Prog Neurobiol. 2009, 88(1): 17-31).

Patent CN109574993A discloses a class of compounds with selective 5-hydroxytryptamine reuptake inhibitory activity and/or 5-HT_{1A} receptor agonistic activity, and specifically discloses a substituted pyrimidine piperazine compound having Formula (I) and preparation method thereof.

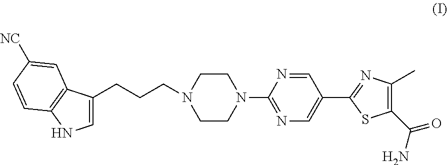

Wherein, the method for preparing the compound having formula (I) is specifically disclosed below:

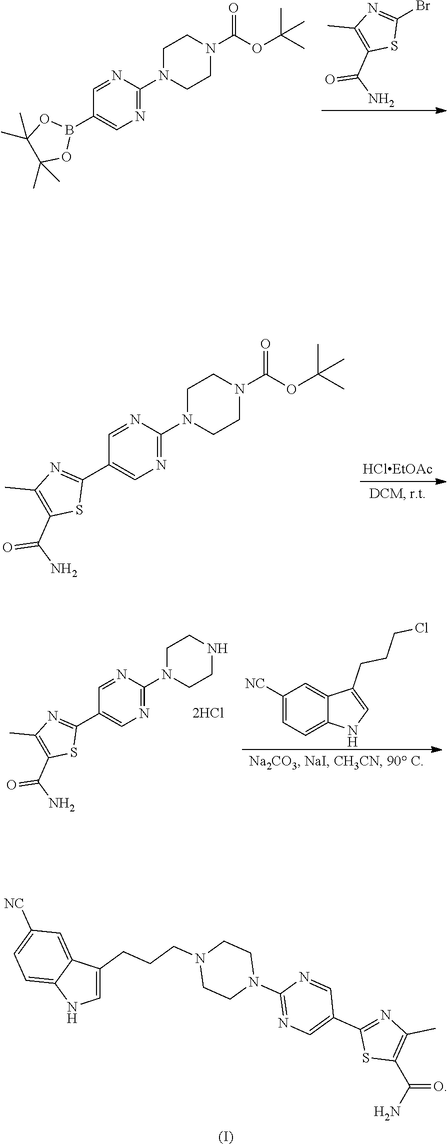

Wherein, in the above method, the amount of the ethyl acetate solution of hydrogen chloride is relatively large (about 10.0 times equivalent), and it is volatile and easily causes air pollution. At the same time, the preparation process is complicated and not suitable for industrial production. Moreover, the reaction time of the last step of the substitution reaction is long (36 hours), which is not suitable for industrial production; further studies have found that the reaction time of the last step of the reaction needs to be further extended when scaled up production, and the reaction process is uncontrollable.

### SUMMARY OF THE INVENTION

In order to overcome the above-mentioned shortcomings in the prior art, provided herein is a new method for preparing the compound having formula (I). This method has the advantages of cheap raw materials, mild conditions, environmental protection, short reaction time, safety and controllability, high total yield, which is especially suitable for industrial production.

In one aspect, provided herein is a new method for preparing the compound having formula (I), comprising:
step 1: in solvent 1, reacting a compound having Formula (II) under the action of base 1 to obtain the compound having Formula (I),

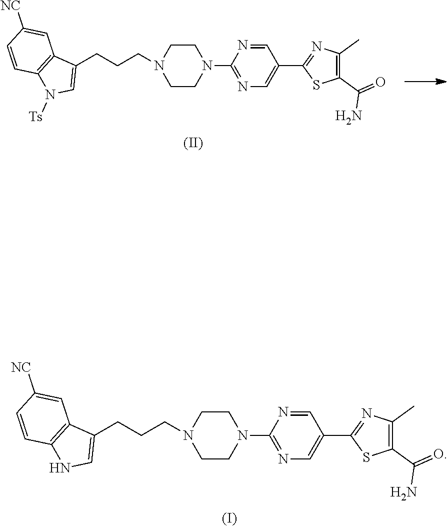 [SMILES: Cc1ccc(S(=O)(=O)n2cc(CCCN3CCN(c4ncc(-c5nc(C)c(C(N)=O)s5)cn4)CC3)c3cc(C#N)ccc32)cc1 ; "(II)" ; Cc1nc(-c2cnc(N3CCN(CCCc4c[nH]c5ccc(C#N)cc45)CC3)nc2)sc1C(N)=O ; "(I)"]

In some embodiments, the base 1 is sodium hydroxide or cesium carbonate.

In other embodiments, the amount of base 1 is multiple times equivalent of the compound having Formula (II); in still other embodiments, the amount of base 1 is 1.0-3.0 times equivalent of the compound having Formula (II).

In some embodiments, the solvent 1 is an alcohol solvent. In other embodiments, the solvent 1 is methanol, ethanol, isopropanol or combinations thereof.

In some embodiments, the reaction of the step 1 is carried out at a certain reaction temperature; in other embodiments, the reaction temperature is 40° C. to 65° C.; in other embodiments, the reaction temperature is 40° C. to 60° C.; in other embodiments, the reaction temperature is 50° C. to 65° C.; in other embodiments, the reaction temperature is 40° C., 50° C., 60° C. or 65° C.

In some embodiments, the method for preparing the compound having formula (I) provided herein further includes a method for preparing the compound having formula (II), comprising:
step 2: in solvent 2, reacting a compound having Formula (III) and a compound having Formula (IV) under the action of base 2 and a catalyst to obtain the compound having Formula (II),

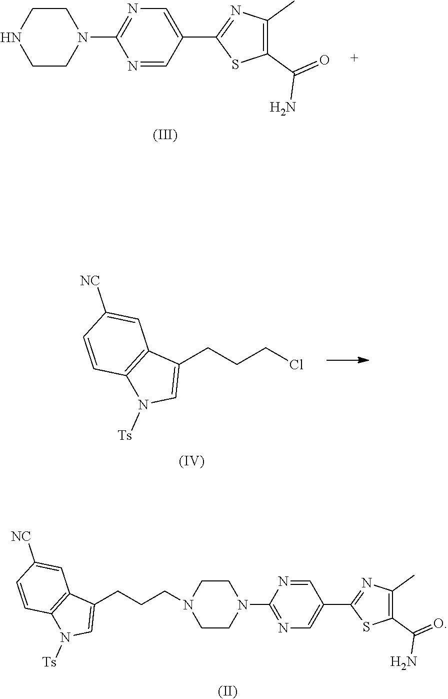 [SMILES: Cc1nc(-c2cnc(N3CCNCC3)nc2)sc1C(N)=O ; "(III)" ; Cc1ccc(S(=O)(=O)n2cc(CCCCl)c3cc(C#N)ccc32)cc1 ; "(IV)" ; Cc1ccc(S(=O)(=O)n2cc(CCCN3CCN(c4ncc(-c5nc(C)c(C(N)=O)s5)cn4)CC3)c3cc(C#N)ccc32)cc1 ; "(II)"]

In other embodiments, the catalyst used in step 2 is sodium iodide or potassium iodide.

In other embodiments, the amount of catalyst is 0.1-0.4 times equivalent of the compound having Formula (III); preferably, the amount of catalyst is 0.2 times equivalent of the compound having Formula (III). In other embodiments, the amount of catalyst is 0.1-0.2 times equivalent of the compound having Formula (III).

In other embodiments, the solvent 2 is N-methylpyrrolidone or N,N-dimethylformamide.

In other embodiments, the base 2 is sodium carbonate or potassium carbonate.

In other embodiments, the amount of base 2 is multiple times equivalent of the compound having Formula (III); in still other embodiments, the amount of base 2 is 2.0-12.0 times equivalent of the compound having Formula (III); in still other embodiments, the amount of base 2 is 2.0-6.0 times equivalent of the compound having Formula (III); in still other embodiments, the amount of base 2 is 2.0-4.0 times equivalent of the compound having Formula (III); in still other embodiments, the amount of base 2 is 3.0-4.0 times equivalent of the compound having Formula (III); in still other embodiments, the amount of base 2 is 2.0 times equivalent, 3.0 times equivalent, 3.5 times equivalent, 4.0 times equivalent, 4.5 times equivalent, 5.0 times equivalent or 6.0 times equivalent of the compound having Formula (III).

In other embodiments, the reaction in step 2 is carried out at a certain reaction temperature; in still other embodiments, the reaction temperature in step 2 is 60° C. to 90° C. In still other embodiments, the reaction temperature in step 2 is 70° C. to 90° C.; in still other embodiments, the reaction temperature in step 2 is 75° C. to 90° C.; in still other embodiments, the reaction temperature in step 2 is 75° C.; in still other embodiments, the reaction temperature in step 2 is 80° C.; in still other embodiments, the reaction temperature in step 2 is 90° C.

In other embodiments, the reaction time of step 2 is 2-8 hours; in still other embodiments, the reaction time of step 2 is 4-6 hours; in still other embodiments, the reaction time of step 2 is 4 hours; in still other embodiments, the reaction time of step 2 is 5 hours; in still other embodiments, the reaction time of step 2 is 6 hours.

In some embodiments, the method for preparing the compound having formula (I) provided herein further includes a method for preparing the compound having formula (III), comprising:
step 3: in solvent 3, reacting a compound having Formula (V) under the action of acid to remove the protective group to obtain the compound having Formula (III),

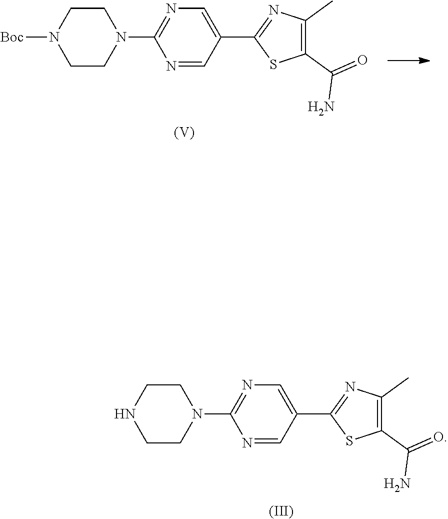 [SMILES: Cc1nc(-c2cnc(N3CCN(C(=O)OC(C)(C)C)CC3)nc2)sc1C(N)=O ; "(V)" ; Cc1nc(-c2cnc(N3CCNCC3)nc2)sc1C(N)=O ; "(III)"]

In other embodiments, the solvent 3 is dichloromethane.

In other embodiments, the acid is sulfuric acid, p-toluenesulfonic acid, phosphoric acid or a solution thereof. Wherein, the acid can be prepared as a solution. Generally, a solvent inert to the reaction in step 3 is selected for preparation. The solution includes, but is not limited to, an ethyl acetate solution of acid.

In other embodiments, the acid is an ethyl acetate solution of sulfuric acid, an ethyl acetate solution of p-toluenesulfonic acid, or an ethyl acetate solution of phosphoric acid.

In other embodiments, the amount of acid is multiple times equivalent of the compound having Formula (V). In still other embodiments, the amount of acid is 2.0-6.0 times equivalent of the compound having Formula (V); preferably, the amount of acid is 3.0-6.0 times equivalent of the compound having Formula (V).

In other embodiments, the reaction in step 3 is carried out in the presence of water.

In still other embodiments, the amount of water is 1.0-4.0 times equivalent of the compound having Formula (V).

In still other embodiments, the amount of acid is multiple times equivalent of the compound having Formula (V). In still other embodiments, the amount of acid is 2.0-6.0 times equivalent of the compound having Formula (V); preferably, the amount of acid is 2.5-4.0 times equivalent of the compound having Formula (V); more preferably, the amount of acid is 3.0 times equivalent of the compound having Formula (V).

Preferably, in the step 3, the compound having formula (V) is reacted to remove the protective group under the action of sulfuric acid to obtain the compound having formula (III) in the presence of water. Wherein, the sulfuric acid can be prepared as an ethyl acetate solution of sulfuric acid. The concentration of the ethyl acetate solution of sulfuric acid can be selected according to the situation. The amount of acid is multiple times equivalent of the compound having Formula (V); preferably, the amount of acid is 2.0-6.0 times equivalent of the compound having Formula (V); more preferably, the amount of acid is 2.5-4.0 times equivalent of the compound having Formula (V); particularly preferably, the amount of acid is 3.0 times equivalent of the compound having Formula (V). The amount of water is multiple times equivalent of the compound having Formula (V); preferably, the amount of water is 1.0-4.0 times equivalent of the compound having Formula (V); more preferably, the amount of water is 2.0 times equivalent of the compound having Formula (V).

In other embodiments, the reaction in step 3 is carried out at room temperature.

On the other hand, provided herein is an intermediate used to prepare the compound having Formula (I), which has a structure as shown in Formula (II):

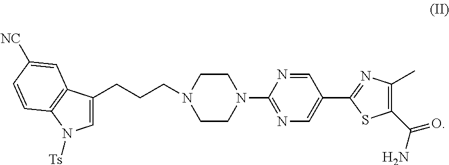 [SMILES: Cc1ccc(S(=O)(=O)n2cc(CCCN3CCN(c4ncc(-c5nc(C)c(C(N)=O)s5)cn4)CC3)c3cc(C#N)ccc32)cc1]

The present invention provides a new method for preparing the compound having Formula (I), which has the following unexpected technical advantages:
1. The compound having Formula (III) has stable properties, and the preparation process is simple and environmentally friendly;
2. The reaction time is greatly shortened, and the yield is high.

Therefore, the new method for preparing the compound having Formula (I) provided herein is more suitable for industrial production.

### DETAILED DESCRIPTION OF THE INVENTION

### Definitions and General Terminology

Reference will now be made in detail to certain embodiments of the invention, examples of which are illustrated in the accompanying structures and formulas. The invention is intended to cover all alternatives, modifications, and equivalents which may be included within the scope of the present invention as defined by the claims. One skilled in the art will recognize many methods and materials similar or equivalent to those described herein, which could be used in the practice of the present invention. The present invention is in no way limited to the methods and materials described herein. In the event that one or more of the incorporated literature, patents, and similar materials differs from or contradicts this application, including but not limited to defined terms, term usage, described techniques, or the like, this application controls.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

Unless defined otherwise, all technical and scientific terms used herein have the same meaning as is commonly understood by one skilled in the art to which this invention belongs. All patents and publications referred to herein are incorporated by reference in their entirety.

As used herein, the following definitions shall apply unless otherwise indicated. For purposes of this invention, the chemical elements are identified in accordance with the Periodic Table of the Elements, CAS version, and the Handbook of Chemistry and Physics, 75th Ed. 1994. Additionally, general principles of organic chemistry are described in “Organic Chemistry”, Thomas Sorrell, University Science Books, Sausalito: 1999, and “March's Advanced Organic Chemistry” by Michael B. Smith and Jerry March, John Wiley & Sons, New York: 2007, the entire contents of which are hereby incorporated by reference.

The grammatical articles “a”, “an” and “the”, as used herein, are intended to include “at least one” or “one or more” unless otherwise indicated herein or clearly contradicted by the context. Thus, the articles used herein refer to one or more than one (i.e. at least one) articles of the grammatical objects. By way of example, “a component” means one or more components, and thus, possibly, more than one component is contemplated and may be employed or used in an implementation of the described embodiments.

In context, all numbers disclosed herein are approximate values. Each numerical value may have a difference of 1%, 2%, 5%, 7%, 8% or 10%. When a number with an N value is made public, any number within N+/−1%, N+/−2%, N+/−3%, N+/−5%, N+/−7%, N+/−8%, or N+/−10% is opened clearly, wherein “+/−” means plus or minus. Whenever a numerical range with a lower limit, DL, and an upper limit, DU, is disclosed, any number falling within the range is specifically disclosed.

The “product content” or “product ratio” in the present invention refers to the content of the product in the reaction system detected by HPLC after the reaction is completed.

In the present invention, “room temperature” refers to a temperature from about 10° C. to about 40° C. In some embodiments, “room temperature” refers to a temperature from about 20° C. to about 30° C.; in other embodiments, “room temperature” refers to 20° C., 22.5° C., 25° C., 27.5° C., and the like.

### EXAMPLES

### Example 1 Synthesis of 4-methyl-2-(2-(piperazin-1-yl)pyrimidin-5-yl)thiazole-5-carboxamide

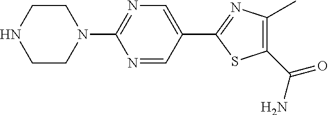 [SMILES: Cc1nc(-c2cnc(N3CCNCC3)nc2)sc1C(N)=O]

Acid was slowly added to ethyl acetate (50 mL), the resulting solution was ready for use. Tert-butyl 4-(5-(5-carboxamido-4-methylthiazol-2-yl)pyrimidin-2-yl)piperazine-1-carboxylate (10.0 g, 24.72 mmol) (for the preparation method, see patent application CN109574993A), dichloromethane (100 mL) and water were added to a reactor, then the ethyl acetate solution of acid was added. After reacting at room temperature for 12 hours, samples were taken and sent to HPLC, the remaining mixed solution was filtered with suction, the filter cake was placed at 50° C. for vacuum drying and dried to obtain a light yellow solid. The experimental results of test examples 1-12 are shown in Table A.

TABLE A

| | Amount | | Amount | Product |
|---|---|---|---|---|
| Test examples | of water | Acid | of acid | content |
| Test example 1 | N/A | p-Toluenesulfonic acid | 6.0 times equivalent | 99.03% |
| Test example 2 | N/A | Trifluoroacetate | 6.0 times equivalent | 0.69% |
| Test example 3 | N/A | Acetic acid | 6.0 times equivalent | 0.08% |
| Test example 4 | N/A | Phosphoric acid | 6.0 times equivalent | 88.90% |
| Test example 5 | N/A | Sulfuric acid | 6.0 times equivalent | 82.31% |
| Test example 6 | N/A | Sulfuric acid | 2.0 times equivalent | 75.99% |
| Test example 7 | N/A | Sulfuric acid | 3.0 times equivalent | 76.09% |
| Test example 8 | 1.0 times equivalent | Sulfuric acid | 3.0 times equivalent | 92.61% |
| Test example 9 | 2.0 times equivalent | Sulfuric acid | 3.0 times equivalent | 98.17% |
| Test example 10 | 4.0 times equivalent | Sulfuric acid | 3.0 times equivalent | 98.56% |
| Test example 11 | 2.0 times equivalent | Sulfuric acid | 2.5 times equivalent | 94.36% |
| Test example 12 | 2.0 times equivalent | Sulfuric acid | 4.0 times equivalent | 98.55% |

Note:
“N/A” refers to the material was not used in the reaction of the example.
“Equivalent” refers to the multiple of the molar amount of the material relative to the reaction substrate tert-butyl 4-(5-(5-carboxamido-4-methylthiazol-2-yl)pyrimidin-2-yl)piperazine-1-carboxylate, for example, 2.0 times equivalent, which means that the molar amount of the material is 2 times that of the reaction substrate tert-butyl 4-(5-(5-carboxamido-4-methylthiazol-2-yl)pyrimidin-2-yl)piperazine-1-carboxylate.

### Test Example 13

Sulfuric acid (1.82 kg, 18.54 mol) was slowly added to ethyl acetate (12.5 L), the resulting solution was ready for use. Tert-butyl 4-(5-(5-carboxamido-4-methylthiazol-2-yl)pyrimidin-2-yl)piperazine-1-carboxylate (2.50 kg, 6.18 mol), dichloromethane (25 L) and water (222 g, 12.36 mol) were added to a reaction kettle, and the ethyl acetate solution of sulfuric acid was added. After reacting at room temperature for 12 hours, samples were taken and sent to HPLC. The product content was 97.94%. The reaction was stopped, the resulting solution was filtered with suction, the filter cake was collected. The filter cake, water (38 L) and sodium carbonate (3.93 kg, 37.08 mol) were added into the reaction kettle, the mixture was stirred at room temperature for 2 hours, filtered with suction, and the filter cake was vacuum dried at 50° C. to obtain a light yellow solid (1.86 kg, yield: 98.9%).

Characterization Data:

MS (ESI, pos. ion) m/z: 305.10 [M+H]⁺;

¹H NMR (400 MHz, D₂O) δ (ppm): 8.36 (d, J=39.5 Hz, 2H), 3.98 (t, J=18.6 Hz, 4H), 3.29 (d, J=4.7 Hz, 4H), 2.30 (d, J=23.8 Hz, 3H).

### Example 2 Synthesis of 2-(2-(4-(3-(5-cyano-1-p-toluene sulfonyl-1H-indol-3-yl)propyl)piperazin-1-yl)pyrimidin-5-yl)-4-methylthiazole-5-carboxamide

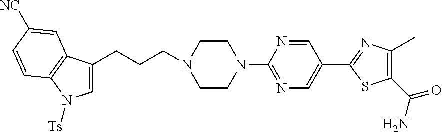 [SMILES: Cc1ccc(S(=O)(=O)n2cc(CCCN3CCN(c4ncc(-c5nc(C)c(C(N)=O)s5)cn4)CC3)c3cc(C#N)ccc32)cc1]

4-Methyl-2-(2-(piperazin-1-yl)pyrimidin-5-yl)thiazole-5-carboxamide (2.0 g, 6.57 mmol), 3-(3-chloropropyl)-1-p-toluenesulfonyl-1H-indole-5-carbonitrile (2.45 g, 6.57 mmol), solvent (20 mL), a certain amount of alkali and sodium iodide (197 mg, 1.31 mmol) were added to a reactor, the mixture was heated to a certain temperature and stirred for 4 hours, samples were taken and sent to HPLC. The reaction was stopped, the resulting solution was filtered with suction. The filtrate was added to the reaction flask, and water (10 mL) was slowly added, the mixture was stirred at room temperature for 4 hours and filtered with suction. The filter cake was washed with water (20 mL) and was vacuum dried at 60° C. to obtain a yellow solid. The experimental results of test examples 1-14 are shown in Table B.

TABLE B

| Test | Reaction | | Amount | Reaction | Product |
|---|---|---|---|---|---|
| examples | temperature | Alkali | of alkali | solvent | content |
| Test example 1 | 90° C. | Potassium carbonate | 4.0 times equivalent | N,N- | 71.5% |
| | | | | dimethylformamide | |
| Test example 2 | 90° C. | Sodium carbonate | 4.0 times equivalent | N,N- | 72.01% |
| | | | | dimethylformamide | |
| Test example 3 | 90° C. | Triethylamine | 4.0 times equivalent | N,N- | 40.64% |
| | | | | dimethylformamide | |
| Test example 4 | 90° C. | N,N- | 4.0 times equivalent | N,N- | 50.28% |
| | | diisopropylethylamine | | dimethylformamide | |
| Test example 5 | 90° C. | Sodium carbonate | 4.0 times equivalent | N,N- | 72.01% |
| | | | | dimethylformamide | |
| Test example 6 | 90° C. | Sodium carbonate | 4.0 times equivalent | N-methylprrolidone | 88.04% |
| Test example 7 | 90° C. | Sodium carbonate | 4.0 times equivalent | acetonitrile | 13.27% |
| Test example 8 | 90° C. | Sodium carbonate | 4.0 times equivalent | acetone | 15.61% |
| Test example 9 | 60° C. | Sodium carbonate | 4.0 times equivalent | N-methylprrolidone | 24.93% |
| Test example 10 | 70° C. | Sodium carbonate | 4.0 times equivalent | N-methylprrolidone | 76.74% |
| Test example 11 | 75° C. | Sodium carbonate | 4.0 times equivalent | N-methylprrolidone | 87.28% |
| Test example 12 | 80° C. | Sodium carbonate | 4.0 times equivalent | N-methylprrolidone | 86.48% |
| Test example 13 | 75° C. | Sodium carbonate | 3.0 times equivalent | N-methylprrolidone | 84.38% |
| Test example 14 | 75° C. | Sodium carbonate | 2.0 times equivalent | N-methylprrolidone | 83.28% |

Note:
“Equivalent” refers to the multiple of the molar amount of the material relative to the reaction substrate 4-methyl-2-(2-(piperazin-1-yl)pyrimidin-5-yl)thiazole-5-carboxamide. For example, 2.0 times equivalent, which means that the molar amount of the material is 2 times that of the reaction substrate 4-methyl-2-(2-(piperazin-1-yl)pyrimidin-5-yl)thiazole-5-carboxamide.

The 4-methyl-2-(2-(piperazin-1-yl)pyrimidin-5-yl)thiazole-5-carboxamide (1.86 kg, 6.11 mol) prepared according to the method of test example 13 in Example 1, 3-(3-chloropropyl)-1-p-toluene sulfonyl-1H-indole-5-carbonitrile (2.28 kg, 6.11 mol), N-methylpyrrolidone (18.6 L), sodium carbonate (2.59 kg, 24.44 mol)) and sodium iodide (0.18 kg, 1.22 mol) were added to a reaction kettle, the mixture was stirred at 75° C. for 6 hours, samples were taken and sent to HPLC, the product content was 89.58%. The reaction was stopped, the resulting solution was filtered with suction. The filtrate was added to the reaction kettle, and water (9 L) was slowly added, the mixture was stirred at room temperature for 4 hours and then filtered with suction. The filter cake was beaten with water (19 L) at room temperature for 2 hours, filtered with suction, and the filter cake was vacuum dried at 60° C. to obtain a yellow solid (3.32 kg, yield: 85.1%).

Characterization Data:

MS (ESI, pos. ion) m/z: 641.15 [M+H]⁺;

¹H NMR (400 MHz, DMSO-d₆) δ (ppm): 8.83 (s, 2H), 8.23 (s, 1H), 8.08 (d, J=8.6 Hz, 1H), 7.90 (d, J=8.3 Hz, 2H), 7.80 (s, 1H), 7.74 (d, J=8.6 Hz, 1H), 7.58 (s, 2H), 7.40 (d, J=8.2 Hz, 2H), 3.79 (brs, 4H), 2.72 (t, J=7.3 Hz, 2H), 2.60 (s, 3H), 2.39 (brs, 4H), 2.32 (s, 3H), 2.29 (d, J=7.2 Hz, 2H).

### Example 3 Synthesis of 2-(2-(4-(3-(5-cyano-1H-indol-3-yl)propyl)piperazin-1-yl)pyrimidin-5-yl)-4-methylthiazole-5-formamide

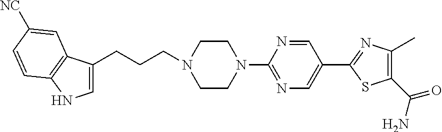 [SMILES: Cc1nc(-c2cnc(N3CCN(CCCc4c[nH]c5ccc(C#N)cc45)CC3)nc2)sc1C(N)=O]

2-(2-(4-(3-(5-cyano-1-p-toluene sulfonyl-1H-indol-3-yl)prop yl)piperazin-1-yl)pyrimidin-5-yl)-4-methylthiazole-5-carboxamide (2.00 g, 3.12 mmol), solvent (20 mL) and a certain amount of alkali were added to a reactor, the mixture was heated to a certain temperature and stirred for 5 hours. Samples were taken and sent to HPLC, and the reaction was stopped, the resulting solution was cooled to room temperature, then filtered with suction, the filter cake was washed with water (10 mL), and the filter cake was vacuum dried at 60° C. to obtain a pale yellow solid. The experimental results of test examples 1-12 are shown in Table C.

TABLE C

| | Reaction | Reaction | | Amount | Product |
|---|---|---|---|---|---|
| Test examples | temperature | solvent | Alkali | of alkali | content |
| Test example 1 | 60° C. | methanol | Cesium Carbonate | 2 times equivalent | 96.42% |
| Test example 2 | 60° C. | ethanol | Cesium Carbonate | 2 times equivalent | 93.65% |
| Test example 3 | 60° C. | i-propanol | Cesium Carbonate | 2 times equivalent | 74.99% |
| Test example 4 | 60° C. | methanol | Sodium hydroxide | 2 times equivalent | 96.34% |
| Test example 5 | 60° C. | methanol | Cesium Carbonate | 2 times equivalent | 96.42% |
| Test example 6 | 60° C. | methanol | Sodium carbonate | 2 times equivalent | 15.48% |
| Test example 7 | 40° C. | methanol | Cesium Carbonate | 2 times equivalent | 82.57% |
| Test example 8 | 50° C. | methanol | Cesium Carbonate | 2 times equivalent | 96.95% |
| Test example 9 | 60° C. | methanol | Cesium Carbonate | 2 times equivalent | 96.42% |
| Test example 10 | 65° C. | methanol | Cesium Carbonate | 2 times equivalent | 96.12% |
| Test example 11 | 60° C. | methanol | Cesium Carbonate | 1 time equivalent | 96.37% |
| Test example 12 | 60° C. | methanol | Cesium Carbonate | 3 times equivalent | 96.09% |

Note:
“Equivalent” refers to the multiple of the molar amount of the material relative to the reaction substrate 2-(2-(4-(3-(5-cyano-1-p-toluenesulfonyl-1H-indol-3-yl)propyl)piperazin-1-yl)pyrimidin-5-yl)-4-methylthiazole-5-carboxamide. For example, 2.0 times equivalent, which means that the molar amount of the material is 2 times that of the reaction substrate 2-(2-(4-(3-(5-cyano-1-p-toluenesulfonyl-1H-indol-3-yl)propyl)piperazin-1-yl)pyrimidin-5-yl)-4-methylthiazole-5-carboxamide.

### Test Example 13

The 2-(2-(4-(3-(5-cyano-1-p-toluene sulfonyl-1H-indol-3-yl)propyl)piperazin-1-yl)pyrimidin-5-yl)-4-methylthiazole-5-carboxamide (3.30 kg, 5.15 mol) prepared according to the method of test example 15 in Example 2, methanol (33 L) and cesium carbonate (3.36 kg, 10.30 mol) were added to a reaction kettle, the mixture was heated to 60° C. and stirred for 5 hours. Samples were taken and sent to HPLC, and the product content was 97.51%. The reaction was stopped, the resulting solution was cooled to room temperature, filtered with suction, the filter cake was washed with water (33 L) and vacuum dried at 60° C. to obtain a pale yellow solid (2.30 kg, yield: 92.0%).

Characterization Data:

MS (ESI, pos. ion) m/z: 487.90 [M+H]⁺;

¹H NMR (400 MHz, DMSO-d₆) δ (ppm): 11.38 (s, 1H), 8.82 (s, 2H), 8.10 (s, 1H), 7.57 (s, 2H), 7.50 (d, J=8.4 Hz, 1H), 7.40 (d, J=8.4 Hz, 1H), 7.35 (s, 1H), 3.84 (brs, 4H), 2.76 (t, J=7.1 Hz, 2H), 2.60 (s, 3H), 2.43 (brs, 4H), 2.36 (t, J=6.7 Hz, 2H), 1.91-1.75 (m, 2H).

1.-26. (canceled)

27. A method for preparing a compound having Formula (I), comprising:
step 1: in solvent 1, reacting a compound having Formula (II) under the action of base 1 to obtain the compound having Formula (I),

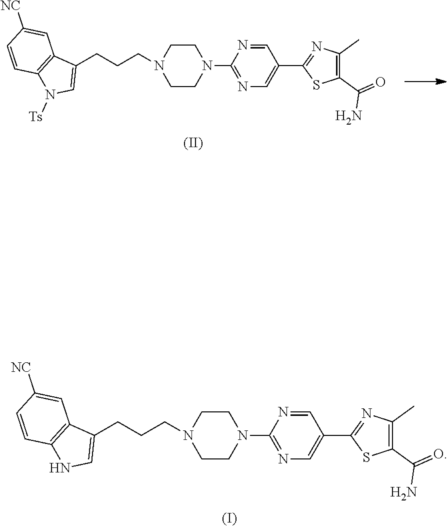 [SMILES: Cc1ccc(S(=O)(=O)n2cc(CCCN3CCN(c4ncc(-c5nc(C)c(C(N)=O)s5)cn4)CC3)c3cc(C#N)ccc32)cc1 ; "(II)" ; Cc1nc(-c2cnc(N3CCN(CCCc4c[nH]c5ccc(C#N)cc45)CC3)nc2)sc1C(N)=O ; "(I)"]

28. The method according to claim 27, wherein the base 1 is sodium hydroxide or cesium carbonate.

29. The method according to claim 27, wherein the amount of base 1 is 1.0-3.0 times equivalent of the compound having Formula (II).

30. The method according to claim 27, wherein the solvent 1 is methanol, ethanol or isopropanol.

31. The method according to claim 27, wherein the reaction temperature of the step 1 is 40° C. to 65° C.; preferably, the reaction temperature of the step 1 is 50° C. to 65° C.

32. The method according to claim 27, which includes a method for preparing the compound having Formula (II), comprising:
step 2: in solvent 2, reacting a compound having Formula (III) and a compound having Formula (IV) under the action of base 2 and a catalyst to obtain the compound having Formula (II),

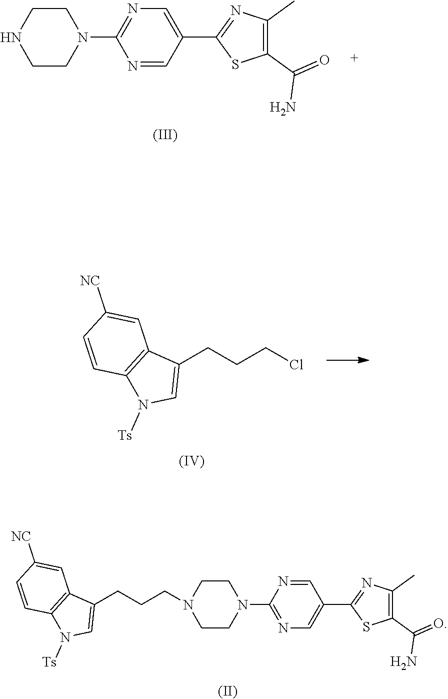 [SMILES: Cc1nc(-c2cnc(N3CCNCC3)nc2)sc1C(N)=O ; "(III)" ; Cc1ccc(S(=O)(=O)n2cc(CCCCl)c3cc(C#N)ccc32)cc1 ; "(IV)" ; Cc1ccc(S(=O)(=O)n2cc(CCCN3CCN(c4ncc(-c5nc(C)c(C(N)=O)s5)cn4)CC3)c3cc(C#N)ccc32)cc1 ; "(II)"]

33. The method according to claim 32, wherein the catalyst is sodium iodide or potassium iodide.

34. The method according to claim 32, wherein the amount of catalyst is 0.1-0.4 times equivalent of the compound having Formula (III); preferably, the amount of catalyst is 0.2 times equivalent of the compound having Formula (III).

35. The method according to claim 32, wherein the solvent 2 is N-methylpyrrolidone or N,N-dimethylformamide.

36. The method according to claim 32, wherein the base 2 is sodium carbonate or potassium carbonate.

37. The method according to claim 32, wherein the amount of base 2 is 2.0-12.0 times equivalent of the compound having Formula (III); preferably, the amount of base 2 is 2.0-4.0 times equivalent of the compound having Formula (III).

38. The method according to claim 32, wherein the reaction temperature in step 2 is 60° C. to 90° C.; preferably, the reaction temperature in step 2 is 70° C. to 90° C.; more preferably, the reaction temperature in step 2 is 75° C.

39. The method according to claim 32, wherein the reaction time in step 2 is 2-8 hours; preferably, the reaction time is 4-6 hours; more preferably, the reaction time is 4 hours, 5 hours or 6 hours.

40. The method according to claim 32, which includes a method for preparing the compound having Formula (III), comprising:
step 3: in solvent 3, reacting a compound having Formula (V) under the action of acid to remove the protective group to obtain the compound having Formula (III), 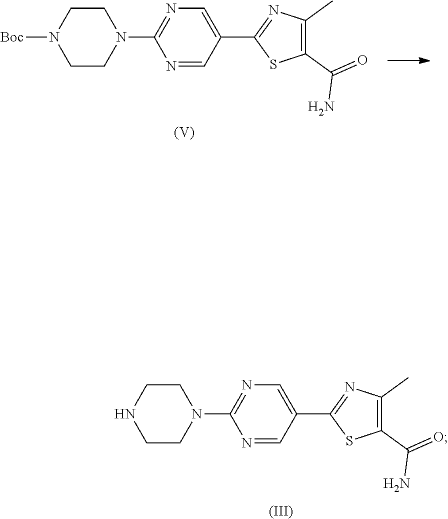 [SMILES: Cc1nc(-c2cnc(N3CCN(C(=O)OC(C)(C)C)CC3)nc2)sc1C(N)=O ; "(V)" ; Cc1nc(-c2cnc(N3CCNCC3)nc2)sc1C(N)=O ; "(III)"]
wherein the solvent 3 is dichloromethane;
wherein the reaction in step 3 is carried out at room temperature.

41. The method according to claim 40, wherein the acid is sulfuric acid, p-toluenesulfonic acid, phosphoric acid or a solution thereof.

42. The method according to claim 40, wherein the acid is an ethyl acetate solution of sulfuric acid, an ethyl acetate solution of p-toluenesulfonic acid, or an ethyl acetate solution of phosphoric acid.

43. The method according to claim 40, wherein the amount of acid is 2.0-6.0 times equivalent of the compound having Formula (V); preferably, the amount of acid is 3.0-6.0 times equivalent of the compound having Formula (V).

44. The method according to claim 40, wherein the reaction in step 3 is carried out in the presence of water; wherein the amount of water is 1.0-4.0 times equivalent of the compound having Formula (V).

45. The method according to claim 44, wherein the amount of acid is 2.0-6.0 times equivalent of the compound having Formula (V); preferably, the amount of acid is 2.5-4.0 times equivalent of the compound having Formula (V); more preferably, the amount of acid is 3.0 times equivalent of the compound having Formula (V).

46. An intermediate used to prepare the compound having Formula (I) as claimed in claim 27, which has a structure as shown in Formula (II): 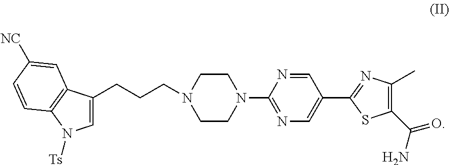 [SMILES: Cc1ccc(S(=O)(=O)n2cc(CCCN3CCN(c4ncc(-c5nc(C)c(C(N)=O)s5)cn4)CC3)c3cc(C#N)ccc32)cc1]
